(19) United States
(12) Patent Application Publication
(10) Pub. No.: US 2015/0374499 A1
(43) Pub. Date: Dec. 31, 2015

(54) FEMORAL PROSTHESIS WITH AN ABSORBABLE SHANK

(71) Applicant: José Roberto ANGELI, (US)

(72) Inventor: José Alberto ANGELI, São Paulo (BR)

(21) Appl. No.: 14/647,968

(22) PCT Filed: Nov. 13, 2013
(86) PCT No.: PCT/BR2013/000489

§ 371 Date: Aug. 1, 2015

(30) Foreign Application Priority Data
Dec. 7, 2012 (BR) 10 2012 031184 4

(51) Int. Cl. A61F 2/36

(52) U.S. Cl. CPC A61F 2/3662; A61F 2/367; A61F 2/3676; A61F 2/3672; A61F 2002/3674; A61F 2002/30062

(57) ABSTRACT

The present subject matter relates to a femoral component of a hip prosthesis consisting of two parts; a superior (metaphyseal) portion made from biocompatible, non-bioabsorbable material, and an inferior (diaphyseal) portion which is bioabsorbable. The diaphyseal portion will be absorbed by the organism months after implantation thereof, which is the necessary time for definitive attachment of the metaphyseal portion in a suitable position by osseointegration. This assembly allows perfect alignment of the prosthesis during the surgical procedure, the diaphyseal shank acting as a guide in the femoral canal, and due to the small size thereof will enable removal of the superior portion if revision surgery is necessary.

The present subject matter relates to a femoral component of a hip prosthesis composed of two parts, a superior (metaphyseal) portion made from biocompatible, non-bioabsorbable material and an inferior (diaphyseal) bioabsorbable portion. The diaphyseal portion will be absorbed by the body months after implantation, which is the necessary time for definitive attachment of the metaphyseal portion in a suitable position by osteointegration. This assembly allows perfect alignment of the prosthesis during the surgical procedure, the diaphyseal shank acting as a guide in the femoral canal, and due to the small size thereof will enable removal of the superior portion if revision surgery is needed.

The present-day hip replacements serve to replace the coxofemoral joint in conditions when the hip has deteriorated due to pain or by fracture.

At present, there are total and partial replacements, where the acetabular portion of the joint may or may not be replaced. The femoral part can be cemented to the bone or not, while prostheses not requiring bone cementing for attachment to the bone are becoming increasingly popular. Although having many advantages, these noncemented prostheses introduce problems which one tries to overcome by various modifications.

Basically, there are two types of femoral components the standard long components, composed of a metaphyseal portion and a diaphyseal portion (modular or single-piece), and the short metaphyseal components, with only the more superior metaphyseal portion.

Of all medical procedures, total arthroplasty of the hip is considered to have the best outcome if measured by patient satisfaction, while the classical prostheses have a shank in the femoral segment which is secured inside the canal of this bone via cement or biological fixation (the bone itself is secured in microporosities on the surface of the prosthesis). This rigid fixation at times produces the following problems:
1. Pain in the thigh due to the difference between the modulus of elasticity of the metal and the bone;
2. Stress shielding, which is bone reabsorption of the proximal region of the femur due to the lack of mechanical stimulus in this region, caused by the transfer of load to the prosthesis and from the latter to the distal part of the femur, making the proximal bone without stimulus more fragile;
3. If it is necessary to remove a fixed prosthesis in a second surgery (revision), this procedure becomes long and difficult, with much bleeding and at times with bone fracture.

To solve these problems, a so-called metaphyseal prosthesis has been created, not having the diaphyseal shank, being fixed only in the proximal (metaphyseal) region of the femur.

Other problems were caused by the lack of the diaphyseal shank:
1. Incorrect positioning by the surgeon, since the diaphyseal shank serves as a guide, orienting the correct positioning of the prosthesis;
2. Problems with the immediate stability of the prosthesis (no press-fit), since the mechanical (not biological) fixation area is very small, which can result in changes in position of the prosthesis and subsequent failure of the surgery;
3. Lack of osseal biological fixation, because the prosthesis is very small and can allow micromovements, preventing growth and fixation of bone tissue on the surface of the prosthesis, also resulting in failure of the surgery.

The present invention overcomes the three aforementioned problems of the traditional prostheses and avoids the three aforementioned problems of the metaphyseal prosthesis while keeping the advantages of the two implants, since it consists of a femoral prosthesis with a diaphyseal shank which can be absorbed by the body, and, since this absorption occurs after the necessary time for the biological fixation, the diaphyseal part will disappear, keeping the metaphyseal part fixed in an adequate position.

The other characteristics of the invention are:
Neck with conical format at its proximal end, enabling the use of femoral heads of different size and material.
Gluing for support on the cortical medial region of the femur, increasing the rotational stability and preventing subsidence of the prosthesis.
Angle on the lateral profile between the metaphyseal and diaphyseal portions adequate to the anatomy of the femoral canal
Threaded hole for fixation between the diaphyseal and metaphyseal portion by use of fastening means
Cavity in the metaphyseal portion allowing a fit of the absorbable part, increasing the rigidity of the system.
Various sizes to adapt to the particular anatomical features of all biotypes.
Wedge-shaped lateral and frontal profiles for stable fit by means of the use of surgical instruments compacting the interior of the femur, giving the canal the same shape as the prosthesis.

For a better explanation of the invention, the figures shall be described:

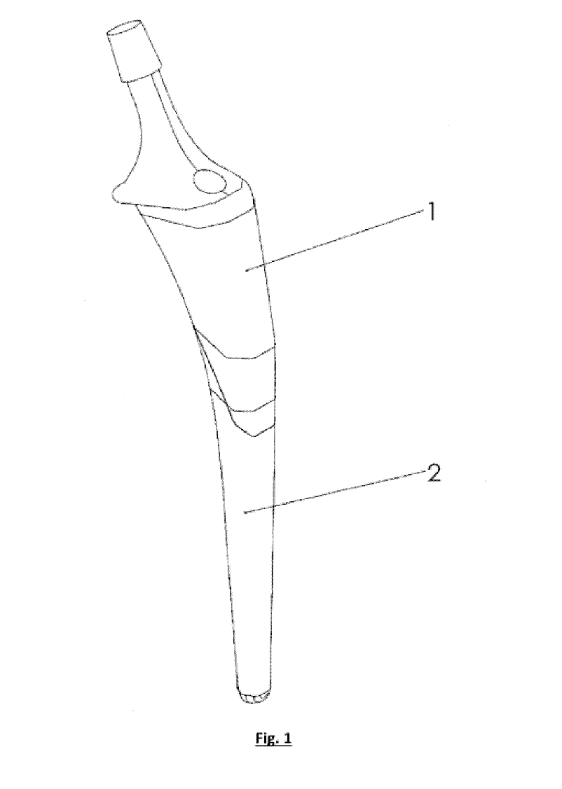

FIG. 1 is a perspective view of the assembled model.

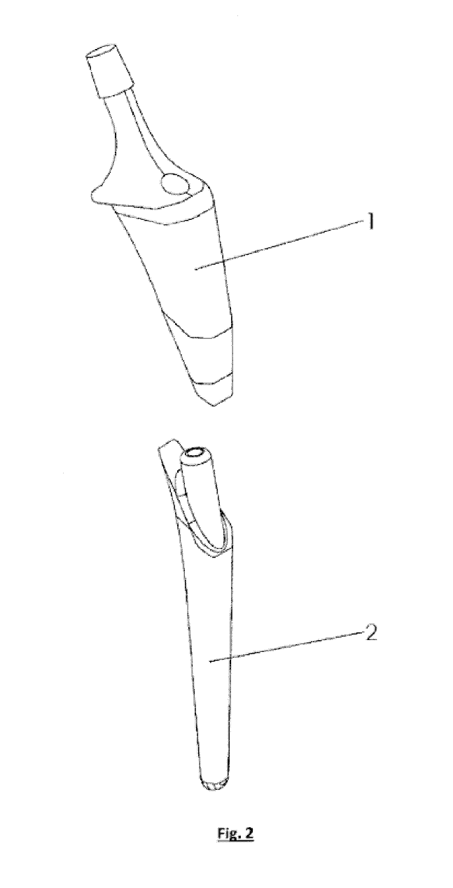

FIG. 2 is a perspective view of the exploded model.

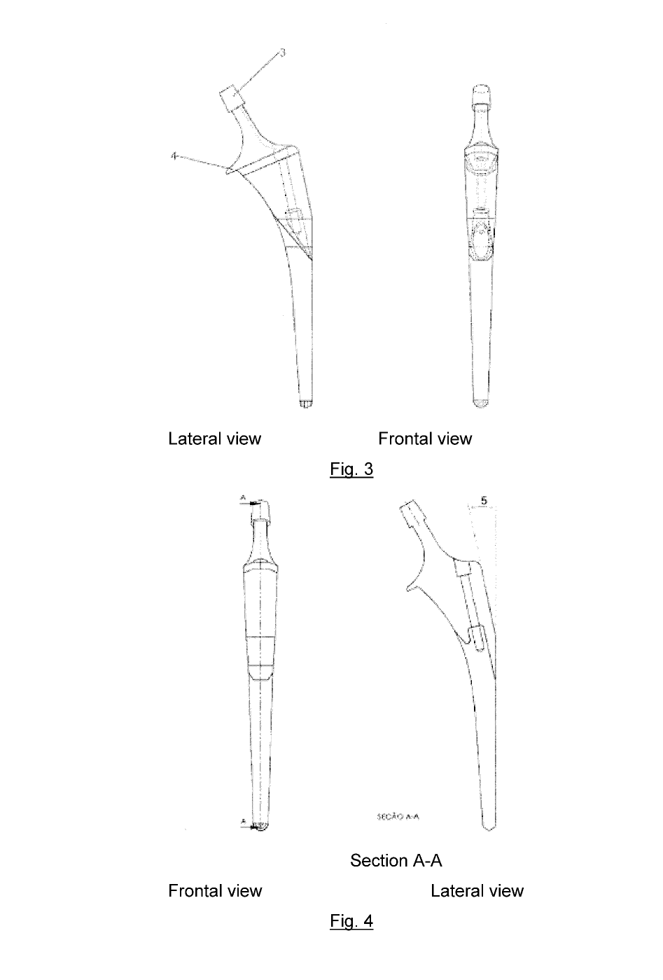

FIG. 3 shows a frontal and lateral view of the metaphyseal and diaphyseal portion joined together.

FIG. 4 is a partial section of the assembly, showing the internal threaded holes of the two parts.

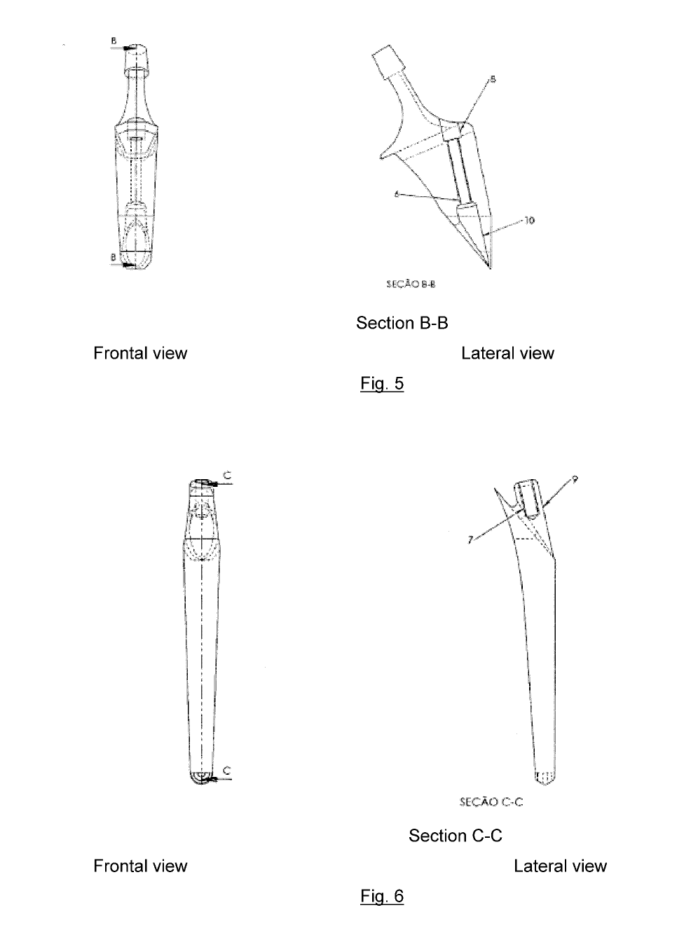

FIG. 5 shows the frontal view of the metaphyseal portion and the lateral sectional view of same.

FIG. 6 shows the frontal view of the diaphyseal portion and the lateral sectional view of same.

According to FIGS. 1 and 2, the present invention is composed of a femoral prosthesis 1, preferably made of metal with a porous and highly biocompatible surface, more preferably coated with a thin layer of hydroxyapatite (artificial bone), its diaphyseal portion 2 being composed of a biocompatible material of slow absorption (such as natural or synthetic polymers, inorganic materials, or combinations of these). As shown in FIG. 3, these two components 1 and 2 are secured together and have a conical neck 3 in the proximal region 1, which allows for the fitting of femoral heads of different size.

Since no cement is used for the fixation, the prosthesis will have different sizes and formats in order to adapt to the anatomy of the most diverse patients and a collar 4 (FIG. 3) for support on the cortical medial region of the femur, increasing the rotational stability and preventing the subsidence of the prosthesis. Its format will preferably be wedge-shaped so that the fitting of the prosthesis in the femoral canal becomes stable. According to FIG. 4, one can observe the angle 5, likewise adequate to the anatomy of the femoral canal.

The fixation between the two segments 1 and 2 will be by fastening means which will be installed in the holes 6 and 7 (FIGS. 5 and 6) or by adhesive materials which are adequate to the stress forces or by means of combinations of the two methods. If it is necessary to remove the metaphyseal portion 1, this hole will have a threaded profile 8 in its superior part which will serve to fit an extractor with its own threaded tip. For all these purposes, the holes 6, 7, 8 will have different diameters along their length, with or without threaded parts.

According to FIG. 6, on the diaphyseal portion 2 there will be a protuberance 9 in the upper part with a threaded hole 7 which will serve to fit and secure the two parts. For the fit, it will be necessary to have a canal 10 with the same geometry as the protuberance in the metaphyseal portion 1.

1. A femoral prosthesis with absorbable shank, characterized by a superior (metaphyseal) portion made of biocompatible material and an inferior (diaphyseal) absorbable portion, fixed together preferably by fixation means and/or adhesive materials.

2. The femoral prosthesis with absorbable shank according to claim 1, further characterized in having different measurements and formats in order to handle different populations and adapt to the femoral canal.

3. The femoral prosthesis with absorbable shank according to claim 1, further characterized by through holes and along the metaphyseal portion, preferably with variations in their diameters, and less preferably without variations in their diameters, for the placement of fixation means and an extractor or positioner when necessary.

4. The femoral prosthesis with absorbable shank according to claim 1, further characterized in having a format in the superior part of the diaphyseal portion which fits into the canal of the metaphyseal portion for the fixation of both parts.
